(19) United States
(12) Patent Application Publication
(10) Pub. No.: US 2002/0160505 A1
(43) Pub. Date: Oct. 31, 2002

(54) Modulation of cellular adhesion with lipid membrane micro-arrays

(76) Inventors: John T. Groves, Berkeley, CA (US); Lara K. Mahal, New York City, NY (US); Carolyn R. Bertozzi, Berkeley, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(21) Appl. No.: 10/076,727

(22) Filed: Feb. 13, 2002

Related U.S. Application Data
(60) Provisional application No. 60/269,625, filed on Feb. 16, 2001.
(60) Provisional application No. 60/296,952, filed on Jun. 8, 2001.

(51) Int. Cl. C12Q 1/00; C12M 1/34

(52) U.S. Cl. 435/287.1; 435/4

(57) ABSTRACT

A method and device for controlled cell adhesion is provided. The device comprises lipid bilayer membranes arranged into discrete areas in a micro-array. They are useful for screening and modulation of living cell adhesion and growth on a solid substrate. The lipid bilayer membranes are doped with various lipids and/or proteins to modulate the adherence of the cells being used in the device.

### CROSS REFERENCE TO RELATED APPLICATIONS

This application claims benefit of provisional application No. 60/269,625, filed Feb. 16, 2001 and entitled “Cell Control With Membrane Micro-Arrays”, and provisional application No. 60/296,952 filed Jun. 8, 2001 and also entitled “Cell Control With Membrane Micro-Arrays”.

### STATEMENT REGARDING FEDERAL FUNDING

This invention was made with U.S. Government support under Contract Number DE-AC03-76SF00098 between the U.S. Department of Energy and The Regents of the University of California for the management and operation of the Lawrence Berkeley National Laboratory. The U.S. Government has certain rights in this invention.

### BACKGROUND OF THE INVENTION

1. Field of the Invention

The present invention relates to cell adhesion assays and, more particularly, to patterned lipid membranes doped with various components to modulate cell adhesion, where the patterned lipid membranes are applied in discrete locations and are supported on a solid support. 2. Description of Related Art

A number of studies have demonstrated that patterning chemical and physical characteristics of a surface can significantly influence cellular behavior. For example, self-assembled monolayers (SAMs) of alkanethiols on gold that present poly-(ethylene glycol) (PEG) moieties effectively resist protein adsorption, thus blocking extracellular matrix (ECM) deposition and subsequent cell adhesion. In contrast, hydrophobic SAMs readily adsorb proteins, including those of the ECM, and consequently promote cell adhesion. Alternatively, an effective surrogate ECM can be provided by chemically coupling an adhesion promoting peptide, such as arginine-glycine-aspartate (RGD), to a silane-modified glass or gold surface (see Massia et al., Anal. Biochem., vol. 187, p. 292 (1990)). Surfaces patterned with adhesive and resistant SAMs, by microcontact printing or photolithography, have been successfully used to guide and control the deposition, growth, and death of cells.

Specific types of devices referred to in the literature are referenced below. A. Lipid devices

Devices containing lipid bilayers patterned in distinct arrays on a solid substrate are disclosed in “Micropatterning Fluid lipid bilayers on solid supports,” Groves et al., Science 275:651-653, Jan. 31, 1997, and Boxer et al. U.S. Pat. No. 6,228,326 B1, “Arrays of Independently-Addressable Supported Fluid Bilayer Membranes,” which is hereby incorporated by reference. These disclosures are directed to a surface detector array wherein the various distinct bilayer surface regions are each coupled to an electrical or optical sensing device. The device is used to sense the binding of an analyte to a “receptor” biomolecule on the biosensor. The receptor biomolecules may be a transmembrane or membrane associated protein that is contained within the lipid bilayer.

Similarly, a fluid lipid bilayer on a glass support patterned with a Ti oxide line projected onto the surface is disclosed in van Oudenaarden and Boxer, “Brownian Ratchets: Molecular Separation in Bilayers Supported on Patterned Arrays, ” Science 285:1046-1048, Aug. 143, 1999.

B. Cell Growth on Lipids

Control of cell adhesion and growth with membrane micro-arrays using phospholipid bilayers is disclosed in Jay T. Groves, et al.; “Control of Cell Adhesion and Growth With Membrane Micro-Arrays”, Biophys. J. (Annual Meeting Abstracts) 2001:144a (Feb. 17, 2001).

C. Patterned Cell Growth

Georger et al. U.S. Pat. No. 5,324,591 discloses a photolithographically defined film for selective cell adhesion, useful in cell culture devices whose surfaces are photolithographically treated.

Kam and Boxer, “Cell Adhesion to protein-micropatterned-supported lipid bilayer membranes,” J. Biomed. Mat. Res. 55:487-495 (Mar. 6, 2001) report a method for constructing controlled interfaces between cells and synthetic supported lipid bilayer membranes. Microcontact printing is used to define squares and grid lines of fibronectin onto glass, which subsequently directs the self-assembly of fluid lipid bilayers onto the complimentary, uncoated regions of the surface.

Earlier references describing procedures of incorporating membrane proteins into supported membranes, include the following: Groves, J. T., Wülfing, C., and Boxer, S. G., Electrical manipulation of glycan-phosphatidyl inositol-tethered proteins in planar supported bilayers, Biophys. J. 1996. 71: 2716-2723; and Salafsky, J., Groves, J. T., and Boxer, S. G., Architecture and function of membrane proteins in planar supported bilayers: a study with photosynthetic reaction centers, Biochemistry 1996, 35, 40: 14773-14781.

References describing biological material microfabrication patterning methods include:

substrate-imposed micropartitioning-Groves, J. T., N. Ulman, and S. G. Boxer, Micropatterning fluid lipid bilayers on solid supports. Science, 1997. 275: p. 651-653, Groves, J. T., N. Ulman, P. S. Cremer, and S. G. Boxer, Substrate-membrane interactions: Mechanisms for imposing patterns on a fluid bilayer membrane. Langmuir, 1998. 14(12): p. 3347-3350, Kung, L., J. T. Groves, N. Ulman, and S. G. Boxer, Printing via photolithography on micropartitioned fluid lipid membranes. Adv. Mater., 2000. 12(10): p. 731-734;

electric field-induced reorganization-Groves, J. T. and S. G. Boxer, Electric field-induced concentration gradients in planar supported bilayers. Biophys. J., 1995, 69: p. 1972-1975; Groves, J. T., C. Wülfing, and S. G. Boxer, Electrical manipulation of glycan-phosphatidyl inositol-tethered proteins in planar supported bilayers. Biophys. J., 1996. 71: p. 2716-2723; Groves, J. T., S. G. Boxer, and H. M. McConnell, Electric field-induced reorganization of two-component supported bilayer membranes. Proc. Natl. Acad. Sci. USA, 1997. 94: p. 13390-13395; Cremer, P. S., J. T. Groves, L. A. Kung, and S. G. Boxer, Writing and erasing barriers to lateral mobility into fluid phospholipid bilayers. Langmuir, 1999.15: p. 3893-3896;

microcontact stamping-Hovis, J. S. and S. G. Boxer, Patterned barriers to lateral diffusion in supported lipid bilayer membranes by blotting and stamping. Langmuir, 2000. 16: p. 894-897.

### SUMMARY OF THE INVENTION

The present invention provides a membrane micro-array for screening and modulation of living cell adhesion and growth on a solid substrate. A number of discrete lipid membranes are arrayed on a solid support. The membranes are disposed on discrete regions termed “corrals,” that may be built into or above an inert, solid substrate. A water layer separates the substrate and the lipid bilayer; this serves to preserve free lateral diffusion of lipids within the membrane and the natural bilayer structure found in cell membranes.

There is lateral diffusion of lipids within each corral, but not between corrals. Different lipid bilayers may be used in different corrals, and are separated by bilayer barrier materials. Bilayer barrier materials include polymers, such as photoresist, metals, such as chrome and gold, and minerals such as aluminum oxide. Alternatively, effective barriers between membrane corrals can be achieved by leaving portions of the substrate free of membrane. The resulting gap serves as a barrier that prevents diffusive mixing between separate corrals.

The substrate of the micro-array preferably comprises any material with a lipid-compatible surface such as SiO₂, MgF₂, CaF₂, mica, polydimethyl siloxane (PDMS), or dextran. SiO₂ is a particularly effective substrate material, and is readily available in the form of glass, quartz, fused silica, or oxidized silicon wafers. These surfaces can be readily created on a variety of substrates, and patterned using a wide range of micro- and nano-fabrication processes including: photolithography, micro-contact printing, electron beam lithography, scanning probe lithography and traditional material deposition and etching techniques.

An important aspect of the present invention is that different corrals may contain different compositions. Some corrals are intended not to permit cell adhesion. Others are intended to promote cell adhesion, or to promote only cell adhesion of certain phenotypes of cells.

In one embodiment, the micro-array comprises a lipid bilayer wherein the primary ingredient is an egg-phosphatidylcholine (PC) membrane. In the absence of dopants, cells do not adhere to this membrane. Other suitable lipids that do not permit cell adhesion include pure phosphatidylcholine membranes such as dimyrstoylphosphatidylcholine or dipalmitoylphosphatidylcholine. Another suitable primary lipid component is phosphatidylethanolamine (PE), which is also, in addition to PC, a primary component.

To achieve cell adhesion, dopants are used. The term “dopant” is used to refer to an “impurity” in the basic lipid composition that is used to alter the properties of the lipid bilayer. The preferred dopant is a negatively, positively or neutrally charged lipid, most preferably the negatively charged lipid phosphatidylserine (PS). Other potential dopants are dipalmitoylphosphatidic acid (PA), distearoylphosphatidylglycerol (PG), phosphatidylinositol, 1,2-dioleoyl-3-dimethylamonnium-propane, 1,2 dioleoyl-3-trimethylammonium-propane (DAP), dimethyldioctadecylammonium bromide (DDAB), 1,2-dioleoyl-sn-glycero-3-ethylphosphocholine (ethyl-PC), N-(7-nitrobenz-2-oxa-1,3-diazol-4-yl)-1,2-dihexadecanoyl-sn-glycero-3-phosphoethanolamine ammonium salt (NDB-PE).

The charges of the above-mentioned lipid dopants are given in Table 1 below. Suitable neutral lipid dopants include cerebrosides and ceramides.

The dopant may also be or include proteins that modulate cell adhesion. The fluid nature of the lipid bilayer of the present invention allows various membrane-bound proteins to be included in the bilayer while retaining their biological activity, including the ability to cluster and move about within the lipid bilayer artificial membrane.

Suitable protein dopants include cell adhesion proteins of the Ig superfamily, e.g. fibronectin, immunoglobin, cadherin, ICAM, N-CAM, C-CAM, etc. as described in Ruoslahti and Obrink, “Common Principles in Cell Adhesion,” Exp. Cell. Res. 227: 1-11 (1996) and Crossin and Krushel, “Cellular Signaling by Neural Cell Adhesion Molecules of the Immunoglobin Superfamily,” Developmental Dynamics, 218:260-279 (2000), both incorporated herein by reference.

Other suitable protein dopants of the Ig superfamily include the major histocompatibility complex proteins, as described e.g. in U.S. Pat .No. 6,232,445 and U.S. Pat. No. 6,309,645, hereby incorporated by reference. The present MHC molecules may include class I and class II human (HLA) peptides and non-human mammalian MHC peptides, as well as individual peptide chains and homo- and hetero-dimers.

Suitable protein dopants also include the selectin family, L-selectin, P-selectin and E-selectin, and the integrin family, LFA-1, α_{L}β₂, Mac-1, α_{M}β₂. p150,95, αₓ,β₂, α_{d}β₂, VLA4, α₄β₁, α₄β₇, etc., as referenced in Krieglstein and Granger, “Adhesion Molecules and Vascular Disease,” Amer. J. Hypertension, 14:445-545, (2001), and Huo and Ley, “Adhesion Molecules and Atherogenesis,” Acta Physiol. Scand., 2001:173:35-43 (2001), both incorporated herein by reference.

Suitable lipid dopants may also include glycolipids such as sphingolipids and other membrane-binding molecules such as cholesterol, or even unknown molecules to be tested. These molecules may be derived synthetically or from cell membranes. For example, to study lymphocyte-endothelial cell interactions, endothelial cell membranes may be fractionated and different fractions added to different corrals in a micro-array. The micro-arrays are then placed in cell culture wells with lymphocytes, and the adhesion of lymphocytes within each well is assessed as to each corral. Adhesion is then correlated with the fraction added to the corral exhibiting membrane-lymphocyte binding, and this fraction may be further fractionated to identify specific adherence-modulating molecules.

The amount of the dopant is selected based on the property of the dopant. For a lipid dopant, 2 to 10%, up to 20% is preferred. This range is also suitable for other non-biologically specific dopants. However, certain receptor proteins, which may be used as dopants in the present invention, are extremely potent when interacting with the appropriate ligand. In this case, as little as 0.00001% of a dopant will be effective.

For example, 10 MHC molecules can trigger adhesion when bound to a single antigen-presenting cell. In the preferred embodiment, there are approximately 5,000 cells that are added to a 10 micron square corral. This results in 1 MHC protein in 1.3×10¹¹ per corral, or 4×10⁻⁶ percent needed to effect cell adherence in the MHC-doped corral.

Another type of dopant is a chemical moiety that chemically binds to a certain type of cell. For example, a hydrazide lipid may be provided as a dopant. It is reactive with ketones, so that it reacts with a ketone molecule that has been engineered to appear on the surface of a cell. This system is described in detail in Bertozzi et al. U.S. Pat. No. 6,075,134, hereby incorporated by reference.

It should also be apparent that dopants may be chosen that block cell adhesion, or that cause behavioral changes in the cells when exposed to the, i.e. doped receptors that trigger morphological changes in the cell, or trigger the expression of pre-determined genes (which may be used to produce signals to detect gene expression).

The choice of dopant will depend on the type of cell and the cellular behavior being tested. In the present work, a fibroblast such as NIH 3T3 cell or a HeLa cervical carcinoma cell were used. In general, any higher eukaryotic cell wherein cell adhesion may be assayed or modulated can be used in the present device and method. Suitable cells and cell lines include, for example, COS-7, HEK 293, BHK and CHO cells.

The present invention also comprises methods for making and using the present devices. The present devices are prepared by:

1. obtaining a substrate, such as a glass wafer

2. partitioning the substrate into corrals, e.g. by depositing micron-scale patterns of chrome;

3. adding one or more dopants to a lipid mixture; and

4. forming lipid bilayers in the individual corrals, e.g. by the vesicle fusion process.

Is it generally necessary to have a water layer between the lipid layer and the substrate, and surrounding the lipid bilayer, to maintain the natural bilayer membrane structure.

In use, the present devices provide a method for screening and modulation of living cell adhesion and growth on a solid substrate, comprising the steps of:

1. adding a micro-array to a cell culture suspension;

2. contacting cells in suspension with a micro-array for a time sufficient for cells to adhere to lipid membranes in the micro-array; and

3. observing resultant cell adhesion or growth.

The present invention also includes an assay to screen and observe differential cell adhesion of living cells to membranes. The assay is performed by: (1) providing a micro-array of membranes in adjacent corrals displayed on a solid substrate, wherein the corrals contain membranes comprised of different compositions of lipids, proteins, and other membrane-associated molecules; (2) contacting and exposing a cell suspension with the membranes displayed on the micro-array and allowing a random diffusion of the living cells; and (3) observing cell adhesion to the membranes over a time period. The membrane composition elements should be sufficiently small to allow the cells to randomly sample many membrane elements before adhering to one. Appropriate membrane elements would be approximately 1 micron to approximately 1 millimeter in size.

Finally, it should be understood that the micro-array of the present invention is used in combination with a cell culture system. This aspect of the invention comprises a system having a multiple well cell culture device; adding a micro-array to each well; adding cells to each well, whereby the cells can move freely within the well to sample different lipid membranes in discreet corrals within the micro-array; maintaining the cells in an uncontaminated culture state; and determining the adherence of cells to particular membranes in multiple corals and in multiple micro-arrays. That is, the method and apparatus of the present invention can be adapted for parallel, high-throughput operation.

### BRIEF DESCRIPTION OF THE DRAWINGS

FIG. 1 is schematic diagram of a supported membrane-containing micro-array device, in top view (FIG. 1A), and side sectional view (FIG. 1B);

FIG. 2 is a photomicrograph of HeLa cells in culture with PS at 6 hours (FIG. 2A), with PS at 24 hours (FIG. 2B), without PS at 6 hours (FIG. 2C), and without PS at 24 hours (FIG. 2D); and

FIG. 3 is a photomicrograph of a micro-array having four corrals. FIG. 3A was taken with fluorescence microscopy, and FIG. 3B was taken with phase contrast microscopy.

### DETAILED DESCRIPTION OF THE PREFERRED EMBODIMENT

I. Introduction

Supported phospholipid membranes present a naturally biofunctional surface that is used in the present method and device to modulate cell behavior. Continuous single bilayer membranes are assembled on solid substrates, such as silica, by spontaneous fusion of lipid bilayer vesicles or by subsequent deposition of two Langmuir monolayers. A thin (˜10 Å) film of water lubricates the membrane-solid interface allowing free lateral diffusion within the fluid lipid bilayer. These membranes are designed to include dopants that modulate cell adhesion and growth characteristics. These dopants may be proteins used to provide an effective artificial cell surface, such as T-lymphocytes or neutrophils. The artificial cell surface may then be tested for cell adhesion properties and/or growth properties with a variety of test cells in culture. Numerous different cell surface properties may be modeled in a single micro-array.

In addition to these remarkable capabilities, supported membranes are readily patterned by a variety of microfabrication techniques including substrate-imposed micropartitioning , electric field-induced reorganization, and microcontact stamping.

Described by way of example below is the use of various lipid compositions in supported membranes as a means of controlling the adhesion and growth of cells on solid substrates. Two naturally adherent cell lines, HeLa (human cervical carcinoma) and NIH 3T3 (mouse fibroblast), were cultured on a panel of supported membranes covering a range of lipid compositions and charge densities. All of the membrane compositions examined block cell adhesion except those containing phosphatidylserine (PS). PS is known to promote the pathological adhesion of erythrocytes (abnormally expressing PS in the outer leaflet of their membrane) to endothelium in conditions such as sickle cell disease, falciparum malaria, and diabetes. In our studies, PS-containing membranes strongly promoted adhesion and growth in both cell lines studied. This finding allowed us to use lipid bilayer patterning technology to selectively direct cell adhesion to specified elements in a membrane micro-array.

II. Definitions

The terms below have the following meanings as applied to this invention:

“Barrier material” means a material used to confine a bilayer in a bounded region, or corral. The barrier material can be formed in a variety of physical configurations: above, below, or coincident with the plane of the bilayer membrane-compatible surface. The barrier material serves to define an outer boundary of a bilayer membrane-compatible surface, so that each bilayer membrane can be isolated within the boundary from other bilayer membranes, thus forming on the substrate an addressably located membrane corrals, or wells of potentially the same or different membrane compositions. An expanse of barrier material will not form a diffusible bilayer over an aqueous layer in turn over a substrate surface. The most preferable barrier material is inactive when exposed to lipid vesicles, retaining no vesicles or their components after exposure It is additionally possible to achieve the effect of a bilayer barrier without the use of a specific barrier material by depositing individual corrals with sufficient separation to prevent them from merging with each other.

“Centrifugation” means separation or analysis of substances in a solution as a function of density and density-related molecular weight by subjecting the solution to a centrifugal force generated by high-speed rotation in an appropriate instrument.

“Corral” means a region where a membrane may be placed on membrane compatible material, which is enclosed by a perimeter of barrier material.

“DAP” means 1,2-dioleoyl-3-dimethylammonium-propane.

“DDAB” means dimethyldioctadecylammonium bromide.

“DMEM” means Dulbecco's modified Eagles medium.

“Dopant” has the meaning set forth in the Summary of the Invention.

“FCS” means fetal calf serum.

“FRAP” means fluorescence recovery after photobleaching.

“HeLa” means the naturally adherent human cervical carcinoma cell line.

“NBD-PE” means N-(7-nitrobenz-2-oxa-1,3-diazol4-yl)-1,2-dihexadecanoyl-sn-glycero-3-phosphoethanolamine, ammonium salt.

“NIH 3T3” means the naturally adherent mouse fibroblast cell line.

“Membrane” means a substrate supported lipid bilayer which mimics a real living cell membrane.

“PA” means dipalmitoylphosphatidic acid.

“PBS” means phosphate-buffered saline.

“PC” means L-(αphosphatidylcholine from egg.

“PG” means distearoylphosphatidylglycerol.

“PI” means phosphatidylinositol from soybean.

“PS” means phosphatidylserine from brain.

“TTAP” means 1,2-dioleoyl-3-trimethylammonium-propane.

“Ultracentrifugation” means performing centrifugation in an instrument at high rotation speeds, generally greater than 40,000 revolutions per minute (rpm), generating centrifugal forces of 100,000 gravities, or approximately 10⁶ M/S².

III. Detailed Description of Micro-array Devices

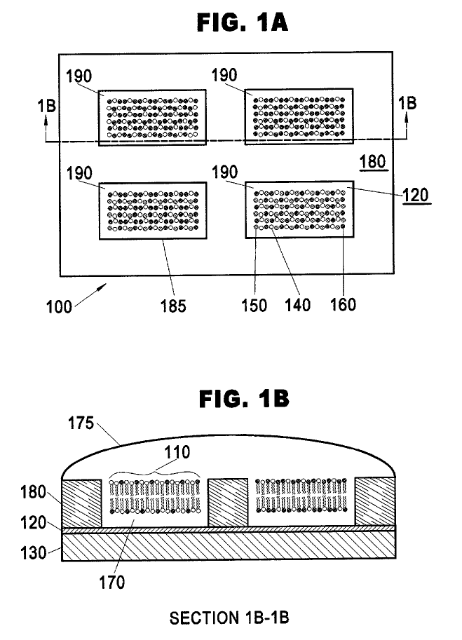

Referring now to FIG. 1, a micro-array device 100 is shown comprising four separate corrals 190. In each corral 190, a lipid bilayer 110 has been formed. The lipid bilayer 110 forms a continuous membrane within the corral 190. Each corral 190 is defined by a barrier material 180 that surrounds the corral and by a surface 120, which is either the same as or is formed upon a substrate 130. The barrier material 180 comprises an inner surface 185 (FIG. 1A) in contact with the lipid bilayer 110 and also forms a physical separation between adjacent corrals 190 and prevents mixing of membrane components between adjacent corrals. The size of the barrier is chosen to allow cells to migrate between corrals and attach (only) to the appropriate lipid membrane, i.e. about 10 microns. The barriers 180 are schematically indicated, but their height has no influence on their function. The barriers 180 preferably do not act like a “cup” that holds liquid by gravity. Surface forces hold the membrane 110 in place, and the topography is largely irrelevant.

Because FIG. 1 is not shown to scale, the various lipid components may be seen. For purposes of illustration, the neutral lipids 140 (open circles) are shown interspersed among negatively charged lipids 150 (shaded circles) or positively charged lipids 160 (dark circles) (FIG. 1A). A layer of water 170 separates the top surface 120 of the substrate 130 from the lipid bilayer 110 in order to permit diffusion within the lipid bilayer 110. While there is water between the membrane 110 and the substrate top surface 120, there is no distinct water surface shown. Membranes 110 can only exist with water on both sides, so the entire membrane is fully submerged at all times, schematically indicated by water droplet 175, which cores all of the corals.

The present micro-array device 100 may also include means for containing cells and cell culture fluid in contact with the corrals 190 by submerging the entire microarray device 100 in a cell culture container (not shown), so that the water, and the culture fluid that it contains are physically restrained and kept sterile.

The present device is preferably made using a silica coverslip or micropatterned chip. This is then placed in a cell culture plate, or other cell culture device.

IV. Making Micro-Array Devices

Planar supported bilayers were formed by fusion of small unilamellar vesicles (SUV) with clean silica substrates (Salafsky, J., J. T. Groves, and S. G. Boxer, Architecture and function of membrane phospholipids in erythrocytes as factor in adherence to endothelial cells in proteins, Biochemistry, 1996, 35: 14773-14781).

A lipid solution in chloroform was evaporated onto the walls of a round bottom flask that was then evacuated overnight. Lipids were resuspended in distilled water by vortexing moderately for several minutes. The lipid concentration at this point was around 3 mg/ml. The lipid dispersion was then probe sonicated to clarity on ice, yielding small unilamellar vesicles (SUV). The SUVs were purified from other lipid structures by ultracentrifugation for 2 hours at 192,000 g. SUVs were stored at 4° C. and typically were stable for a few weeks to several months. L-α phosphatidylcholine from egg (egg-PC), phosphatidylserine from brain (PS), dipalmitoylphosphatidic acid (PA), distearoylphosphatidylglycerol (PG), phosphatidylinositol from soybean (PI), 1,2-dioleoyl-3-dimethylammonium-propane (DAP), 1,2-dioleoyl-3-trimethylammonium-propane (TAP), dimethyldioctadecylammonium bromide (DDAB), 1,2-dioleoyl-sn-glycero-3-ethylphosphocholine (ethyl-PC), N-(7-nitrobenz-2-oxa-1,3-diazol-4-yl)-1,2-dihexadecanoyl-sn-glycero-3-phosphoethanolamine, ammonium salt (NBD-PE) were obtained from Avanti Polar Lipids (Alabaster, Ala.). N-(Texas Red® sulfonyl)-1,2-dihexadecanoyl-sn-glycero-3-phosphoethanolamine, triethylammonium salt (Texas Red DHPE) were obtained from Molecular Probes (Eugene, Oreg.).

Micropatterned substrates were fabricated on borofloat glass wafers (diameter=100 mm; thickness=0.7 mm) from Precision Glass & Optics (Santa Ana, Calif.). A 1000 Å layer of chrome was deposited on the wafers by vapor deposition. The wafers were then spin coated with Shipley positive photoresist and exposed through a photomask (Photosciences, Torrence, Calif.) by contact lithography. The resist was developed and the chrome was etched back with CR-4 chrome etch from Cyantek Corp. (Fremont, Calif). The wafers were then cut into 9 mm square chips that are reusable.

Silica coverslips or micropatterned chips were cleaned by soaking for 20 minutes in freshly prepared piranha solution (3:1 mixture of sulfuric acid and 30% hydrogen peroxide). The substrates were then rinsed under DI water and blown dry with compressed air. Prior to supported membrane deposition, spreading solutions were prepared by mixing the SUV suspensions in equal ratios with PBS. For deposition onto coverslips, a 30 μl drop of membrane spreading solution was placed in the center of a well (i.e. corral) in the cell culture plate and a substrate was immediately laid on top of the drop. Within one minute, the well was filled with PBS. The substrate was then carefully flipped over to expose the membrane surface (membranes must be kept submerged at all times). Each well was flushed several times with PBS to remove excess vesicles then flushed with cell culture media. Array deposition on micropatterned substrates was carried out by direct pipetting (Cremer, P.S. and T. Yang, Creating spatially addressed arrays of planar supported fluid phospholipid membranes. J. Am. Chem. Soc., 1999. 121: 8130-8131).

V. Uses of the Micro-array Devices

NIH 3T3 and HeLa cells were cultured in Dulbecco's modified Eagles medium (DMEM) containing 10% fetal calf serum (FCS). Cells were grown in a 37° C. incubator with 5% CO₂ atmosphere. Cells were washed, trypsinized and resuspended in DMEM with 10% FCS. An average of 200,000 cells were added per sample (˜50,000 cells/cm²).

The cells are then cultured on the supported lipid bilayer membranes. The devices are maintained in sterile conditions by assembling them within a sterile laminar flow hood and following basic sterile procedures as used in traditional cell culture.

The use of the present micro-array device requires that the eukaryotic cells to be tested and to be maintained in a relatively sterile state and under suitable cellular metabolic conditions to promote cell adhesion to the appropriately doped lipid membrane. That is, the cells do not have to be actually dividing, but should be in a growth phase.

Sterile conditions are facilitated because the chip is made in a sterile manner. The substrate manufacturing process sterilizes the inert materials used in making the micro-array. The lipid materials are prepared under sterile conditions. The chip is assembled in a cell culture facility under standard sterile conditions. It can be used in a variety of environments. The most simple environment is simply a Costar® multiple well culture cluster. Costar is a registered trademark of Corning, Inc. The micro-arrays are placed in the bottoms of the wells. The cells settle down on the array and creep around until they find the appropriate anchorage components.

After culture of the cells on the membranes for 1 to 48 hours, detection of cell adhesion is carried out. As described below, this may be by physical shaking of the device, or may be carried out optically, electrically, or by other means.

In some cases, the present micro-array devices may be used in testing the effects of various drugs or growth conditions on selected cells. The cells are incubated under conditions with or without the drug and then plated onto the micro-array. The culture medium of the micro-array may also contain a drug of interest. After observing the growth of the cells on the lipid membranes of the micro-array, conclusions may be drawn about the activity of the drug with respect to the interaction of the test cell with various simulated cell membrane surfaces.

VI. Selection of Materials for Lipid Bilayer Membranes

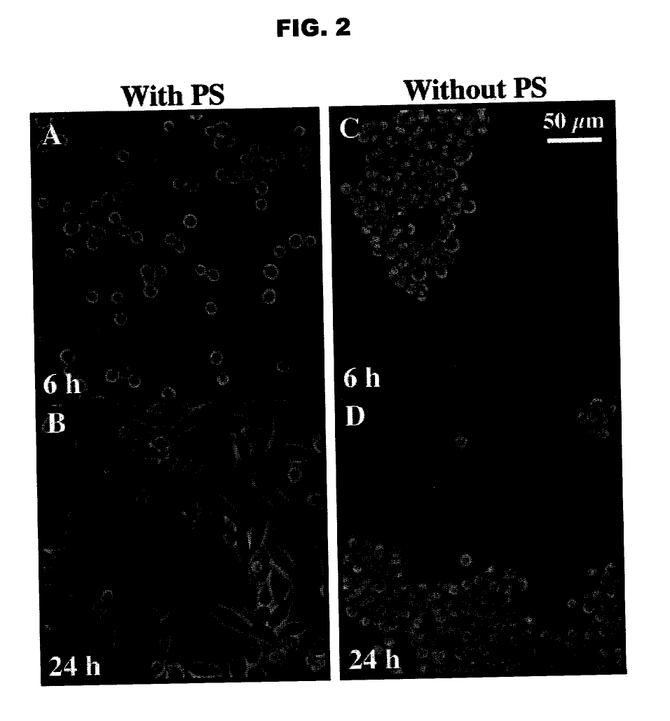

Cell adhesion was characterized on egg-phosphatidylcholine (egg-PC) membranes doped with a variety of negatively and positively charged lipids. Doping ratios of 5 mol % were studied for each of the charged lipids; positively charged DAP and negatively charged PS were also examined at 3 mol % and 9 mol % doping ratios. In all cases, PS containing membranes promoted cell adhesion while other compositions effectively blocked cell adhesion. The results from a typical adhesion assay are illustrated in FIG. 2.

Referring now to FIG. 2, Membranes in 2A and 2B are 5% PS:94% PC:1% NBD-PE; those in C and D are 5% DAP:94% PC:1% NBD-PE. Observations were made at 6 and 24 hours as labeled. Observations as shown in FIG. 2 reveal that initial adhesive contacts between HeLa cells and the substrate formed within 6 hours when the supported membranes contained PS. At this stage, the cells were evenly distributed over the substrate and remained fixed in place under mild shaking. After 24 hours, focal adhesion sites were well formed and cells exhibited morphology consistent with that observed in tissue culture plates. As shown in FIG. 2B, cells began to form typical spindle shapes due to cellular adhesion.

In contrast, membranes lacking PS blocked cell adhesion. After 6 hours, cells tended to clump together and were not fixed to the substrate as determined by their movement under mild shaking. Little or no change in this condition was observed after 24 hours. In all experiments, cells that settled on the plastic tissue culture plate grew normally, thus providing an internal positive control for cell viability. Results for the specific lipids and cells studied are listed in Table 1.

TABLE 1
Cell adhesion data

| Lipid | Charge | Adhesion* | Cell Lines Test |
|---|---|---|---|
| PS | − | yes | HeLa, NIH 3T3 |
| PA | − | no | HeLa |
| PG | − | no | HeLa |
| PI | − | no | HeLa |
| DAP | + | no | HeLa, NIH 3T3 |
| TAP | + | no | HeLa |
| DDAB | + | no | HeLa |
| Ethyl-PC | + | no | HeLa |

*Cell adhesion data was collected on egg-PC supported membranes containing 5 mol % of the charged lipid under study.

The use of PS as the adhesion promoting ligand in fluid membranes creates a number of attractive micropatterning opportunities. For example, the vesicle fusion process can be used to deposit supported membranes in enclosed spaces such as the interior capillary walls of a microfluidic chip. Furthermore, laterally applied electric fields can generate spatial patterns, such as continuous concentration gradients or localized enrichments of the negatively charged PS, which can be reorganized dynamically. PS-mediated control of cells with supported membranes is simple and provides a variety of unique capabilities, which compliment existing cell patterning technologies.

VII. Testing of Membrane Integrity

Lateral fluidity of supported membranes is one of their most distinctive features and can serve as a stringent test of membrane integrity. Long-range mobility of membrane components is easily observed by fluorescence recovery after photobleaching (FRAP). FRAP is based on the principal of observing the rate of recovery of fluorescence due to the movement of a fluorescent marker into an area of the membrane which contains this same marker but which has been rendered non-fluorescent via an intense photobleaching pulse of laser light. The two-dimensional diffusion coefficient (D) of the fluorophore is related to both its rate and extent of recovery. FRAP has proved to be a popular means to assess the structure of artificial and biological membranes.

In the present case, PS-mediated cell adhesion was used to pattern cell growth on supported membrane surfaces. Membrane micro-arrays displaying alternating corrals of PS-containing and PS-free membrane were deposited on pre-fabricated substrates with either 200 μm or 500 μm grid sizes. The membrane within each corral in the micro-array is fluid while grids of chrome barriers on the silica substrate prevent mixing between separate corrals. Different fluorescently labeled lipids were incorporated in the various membrane types, allowing them to be distinguished in the micro-array.

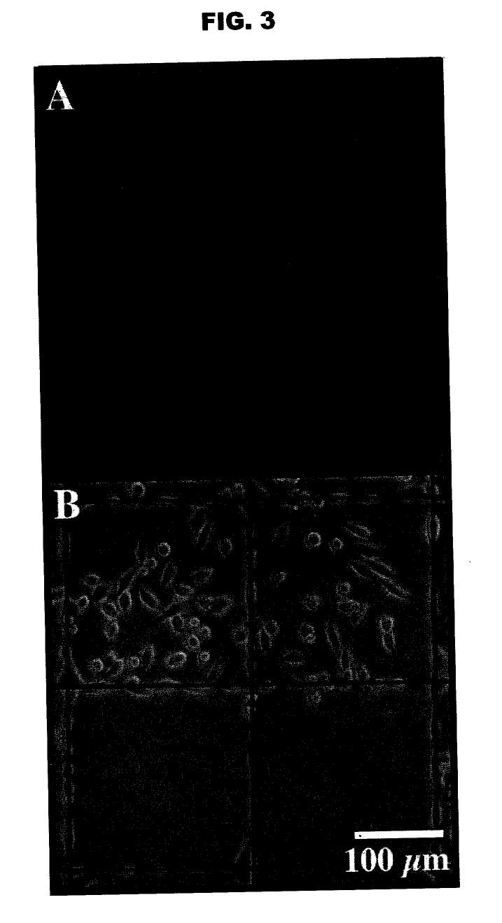

Referring now to FIGS. 3A and 3B, cells cultured on micro-array surfaces selectively adhered to and grew on the PS-containing membrane corrals, which are the top two corrals in FIG. 3A and in FIG. 3B. FIG. 3A illustrates fluorescence and FIG. 3B phase contrast photomicrographs of a 4-corral section of a micro-array; both images were taken 24 hours after cell inoculation. Red fluorescence observed from the membranes in the upper two corrals identifies them as PS-containing (5% PS:94% PC:1% Texas Red-PE) while the lack of membrane fluorescence in the lower two corrals indicated that those membranes are PS-free (5% PG:94% PC:1% NBD-PE). The corresponding phase contrast image of FIG. 3B illustrates the distinct segregation of the HeLa cells onto the two upper PS-containing membrane corrals. The nearly complete lack of cell deposition on the identically charged PG-containing membrane (lower two corrals in FIG. 3B) underscores the chemical specificity of the PS effect. In multiple experiments with various membrane combinations, cells were observed to proliferate to near confluence on the PS-containing membrane while PS-free membrane corrals remain essentially devoid of cells.

Thus the present invention has been described in detail with reference to the presently preferred embodiment of the invention. The true scope of the present invention should be regarded as defined by the appended claims.

What is claimed is:

1. A micro-array device for determining adherence of selected cells contacting the device to a lipid membrane of the device, comprising:
a. an inert solid substrate;
b. a plurality of lipid membranes arrayed on said substrate in physically separate corrals defined by barriers on the substrate, each corral sized to contact a plurality of cells moving from corral to corral;
c. said lipid membranes forming, in each corral, a continuous, fluid sheet; and
d. dopant molecules, present within some but not all membrane sheets of the micro-array, each molecule movable within its sheet, said dopant molecules selected for cell adhesion properties that change the binding property of the lipid membrane towards the selected cell.

2. The micro-array device of claim 1, wherein said dopant molecule is selected from the group consisting of:
a. lipids;
b. cell adhesion proteins of the immunoglobin superfamily; and
c. selecting.

3. The micro-array according to claim 2, further comprising a layer of water separating the substrate from the membranes.

4. The micro-array according to claim 3, further comprising means for immersing the micro-array within a mixture of cells and culture fluid.

5. The micro-array according to claim 1, wherein the membrane lipid is phosphatidylcholine.

6. The micro-array according claim 1, wherein the membrane lipid and/or the dopant is selected from the group consisting of phosphatidylserine, dipalmitoylphosphatidic acid, distearoylphosphatidylglycerol, phosphatidylinositol, 1,2-dioleoyl-3-dimethylammonium-propane, 1,2-dioleoyl-3-trimethylammonium-propane, dimethyldioctadecylammonium bromide, 1,2-dioleoyl-sn-glycero-3-ethylphosphocholine, N-(7-nitrobenz-2-oxa-1,3-diazol-4-yl)-1,2-dihexadecanoyl-sn-glycero-3-phosphoethanolamine ammonium salt, and N-1,2-dihexadecanoyl-sn-glycero-3-phosphoethanolmine triethylammonium salt.

7. A method for screening living cell adhesion on a solid substrate comprising:
a. contacting a cell suspension with a micro-array comprising an array of adjacent membrane corrals, membrane lipids and positively or negatively charged lipids; and
b. observing cell adhesion after a time period of at least one hour.

8. A method for determining the cell adhesion properties of a living adherent cell, comprising:
a. providing a micro-array device having a plurality of lipid bilayer membranes disposed on a solid substrate in corrals separated by a barrier material, said lipid bilayer membranes having different compositions in different corrals;
b. culturing a population of cells in said micro-array device; and
c. determining the adhesion of the cells to the lipid bilayer membranes in different corrals in response to said different compositions.

9. The method according to claim 8, wherein the micro-array comprises membranes supported by a solid substrate, and wherein the membranes are doped with negatively or positively charged lipids.

10. The method according to claim 9, wherein the solid substrate is separated from the membranes by a water layer and further comprising a material separating individual membrane corrals to permit a lateral diffusion of lipids within each corral, thereby enabling use of different membrane compositions for different corrals.

11. The method according to claim 10, wherein the substrate is a micropatterned glass wafer.

12. The method according to claim 11, wherein the membrane is an egg-phosphatidylcholine membrane.

13. The method according to claim 12, wherein the lipid is selected from the group consisting of phosphatidylserine, dipalmitoylphosphatidic acid, distearoylphosphatidylglycerol, phosphatidylinositol, 1,2-dioleoyl-3-dimethylammonium-propane, 1,2-dioleoyl-3-trimethylammonium-propane, dimethyldioctadecylammonium bromide, 1,2-dioleoyl-sn-glycero-3-ethylphosphocholine, N-(7-nitrobenz-2-oxa-1 ,3-diazol-4-yl)-1,2-dihexadecanoyl-sn-glycero-3-phosphoethanolamine ammonium salt, and N-1,2-dihexadecanoyl-sn-glycero-3-phosphoethanolmine triethylammonium salt.

14. An assay to screen and observe differential cell adhesion of living cells to membranes comprising:
a. providing a micro-array of membranes in corrals displayed on a solid substrate, wherein the corrals contain membranes comprised of different compositions of lipids, proteins, and other membrane-associated molecules; then
b. contacting and exposing a cell suspension with the membranes displayed on the micro-array and allowing a random diffusion of the living cells on the membrane; and
c. observing cell adhesion to the membranes over a time period.

15. The assay according to claim 14, wherein the membrane composition elements are sufficiently small to allow the cells to randomly sample many membrane elements before adhering to one.

16. The assay according to claim 15, wherein the membrane elements are approximately 1 micron to approximately 1 millimeter in size.

17. The assay according to claim 16, wherein the solid substrate of the micro-array is separated from the membranes by a water layer and further comprising a material separating membrane corrals, thereby permitting a lateral diffusion of membranes within each corral.

18. The assay according to claim 17, wherein the micro-array substrate is a micropatterned glass wafer.

19. The assay according to claim 17, wherein the lipid is selected from the group consisting of phosphatidylserine, dipalmitoylphosphatidic acid, distearoylphosphatidylglycerol, phosphatidylinositol, 1,2-dioleoyl-3-dimethylammonium-propane, 1,2 dioleoyl-3-trimethylammonium-propane, dimethyldioctadecylammonium bromide, 1 ,2-dioleoyl-sn-glycero-3-ethylphosphocholine, N-(7-nitrobenz-2-oxa-1,3-diazol-4-yl)-1,2-dihexadecanoyl-sn-glycero-3-phosphoethanolamine ammonium salt, and N-1,2-dihexadecanoyl-sn-glycero-3-phosphoethanolmine triethylammonium salt.

20. The assay according to claim 17, wherein the membrane is an egg-phosphatidylcholine membrane.

21. A membrane bilayer surface comprised of:
a plurality of phosphatidylserine-free small unilamellar vesicles that have been deposited on a membrane compatible surface to form a cell adhesion-free phospholipid bilayer surface.

22. The membrane bilayer surface of claim 21 wherein said cell adhesion-free phospholipid bilayer surface is an interior capillary wall of a microfluidic device to provide a cell adhesion-free microfluidic device.

23. A patterned surface comprised of:
a plurality of variously doped and undoped small unilamellar vesicles bonded to a membrane compatible surface in a pattern to form a patterned cell adhesion and non-cell-adhering phospholipid bilayer surface.

24. The patterned surface of claim 23 wherein said patterned cell adhesion and non-cell-adhering phospholipid bilayer surface is further measured to detect the presence, absence, or quantity of cells present on said surface.
